(19) United States
(12) Patent Application Publication
(10) Pub. No.: US 2026/0338972 A1
(43) Pub. Date: Oct. 29, 2026

(54) RADIATION IMAGE DETECTOR

(71) Applicant: FUJIFILM Corporation, Tokyo (JP)

(72) Inventors: Kotatsu KAWAGUCHI, Kanagawa (JP); Takeyasu KOBAYASHI, Kanagawa (JP)

(73) Assignee: FUJIFILM Corporation, Tokyo (JP)

(21) Appl. No.: 19/648,694

(22) Filed: Apr. 15, 2026

(30) Foreign Application Priority Data
Apr. 24, 2025 (JP) 2025-072681

(51) Int. Cl. G01T 1/20; A61B 6/42

(52) U.S. Cl. CPC G01T 1/20189; A61B 6/4233

(57) ABSTRACT

A radiation image detector includes a detection panel that detects a radiation image corresponding to radiation transmitted through a subject, a circuit board on which an electrical circuit component is mounted, and a housing that accommodates the detection panel and the circuit board. The housing includes an accommodation portion in which an opening portion is formed on an incidence side of the radiation, and a radiation transmission member that closes the opening portion and transmits the radiation. The radiation transmission member has a back surface on a side opposite to the incidence side of the radiation, to which the detection panel is attached. A heat insulating member is disposed between the accommodation portion and the radiation transmission member. The heat insulating member insulates drive heat of the electrical circuit component transmitted to the detection panel through the accommodation portion and the radiation transmission member.

### CROSS-REFERENCE TO RELATED APPLICATIONS

The present application claims priority under 35 U.S.C. § 119 to Japanese Patent Application No. 2025-072681, filed on Apr. 24, 2025. The above application is hereby expressly incorporated by reference, in its entirety, into the present application.

### BACKGROUND

### 1. Technical Field

The technology of the present disclosure relates to a radiation image detector.

### 2. Description of the Related Art

A radiation image detector including a detection panel that detects a radiation image according to radiation transmitted through a subject, a circuit board on which an electrical circuit component is mounted, and a housing is known. The housing includes an accommodation portion that accommodates the detection panel and the circuit board and in which an opening portion is formed on an incidence side of the radiation, and a radiation transmission member that closes the opening portion and transmits the radiation.

In the radiation image detector, there is a problem in that the image quality of the radiation image deteriorates due to the influence of drive heat of the electrical circuit component on the detection panel. In order to solve this problem, for example, JP2003-194951A discloses a radiation image detector having a structure in which drive heat of an electrical circuit component is dissipated to a bottom plate of an accommodation portion by a heat radiation member.

### SUMMARY

In the radiation image detector, a detection panel is attached to a back surface of a radiation transmission member. In a case in which such a configuration is adopted, in the technique disclosed in JP2003-194951A, there is a concern that the drive heat of the electrical circuit component is transmitted to the detection panel through the accommodation portion and the radiation transmission member.

One embodiment according to the technology of the present disclosure provides a radiation image detector capable of reducing a concern that drive heat of an electrical circuit component is transmitted to a detection panel through a radiation transmission member.

A radiation image detector according to the present disclosure comprises: a detection panel that detects a radiation image corresponding to radiation transmitted through a subject; a circuit board on which an electrical circuit component is mounted; a housing that includes an accommodation portion that accommodates the detection panel and the circuit board and in which an opening portion is formed on an incidence side of the radiation, and a radiation transmission member that closes the opening portion and transmits the radiation, the detection panel being attached to a back surface of the radiation transmission member on a side opposite to the incidence side; and a heat insulating member that is disposed between the accommodation portion and the radiation transmission member and insulates drive heat of the electrical circuit component transmitted to the detection panel through the accommodation portion and the radiation transmission member.

It is preferable that the heat insulating member is disposed around an entire periphery of the opening portion, and the accommodation portion and the radiation transmission member are completely separated from each other by the heat insulating member.

It is preferable that a first portion of the accommodation portion and a first portion of the radiation transmission member are in contact with each other, and a remaining second portion of the accommodation portion and a remaining second portion of the radiation transmission member are separated from each other by the heat insulating member.

It is preferable that the first portion has a smaller area than the second portion.

It is preferable that the detection panel is composed of a scintillator that converts the radiation into light, and a light detection substrate on which pixels that accumulate electric charges corresponding to the light are arranged, and the light detection substrate and the scintillator are disposed in this order from the incidence side of the radiation.

It is preferable that the accommodation portion further accommodates a middle plate, and the circuit board is attached to a back surface of the middle plate on a side opposite to the incidence side.

It is preferable that the circuit board is attached to the back surface of the middle plate via a spacer, the electrical circuit component is mounted on a surface of the circuit board facing the back surface of the middle plate, and the heat insulating member is disposed between the electrical circuit component and the back surface of the middle plate.

It is preferable that an end portion of the middle plate is attached to an inner surface of a side plate of the accommodation portion.

It is preferable that the heat insulating member is disposed between the end portion of the middle plate and the inner surface of the side plate.

It is preferable that the end portion of the middle plate is attached to an inner surface of a bottom plate of the accommodation portion.

It is preferable that the heat insulating member is disposed between the end portion of the middle plate and the inner surface of the bottom plate.

It is preferable that the heat insulating member has a lower thermal conductivity than the accommodation portion.

It is preferable that the heat insulating member has a thermal conductivity of 0.10 W/(m·K) or less.

According to the technology of the present disclosure, it is possible to provide a radiation image detector capable of reducing a concern that drive heat of an electrical circuit component is transmitted to a detection panel through a radiation transmission member.

### BRIEF DESCRIPTION OF THE DRAWINGS

FIG. 1 is a diagram showing an example of use of a radiation image detector.

FIG. 2 is an external perspective view of the radiation image detector.

FIG. 3 is an exploded cross-sectional view of the radiation image detector.

FIG. 4 is a cross-sectional view of the radiation image detector.

FIG. 5 is a diagram showing a conduction path of drive heat of an electrical circuit component in the related art.

FIG. 6 is a diagram showing a conduction path of drive heat of an electrical circuit component in the technology of the present disclosure.

FIG. 7 is a diagram showing Modification Example 1 of a disposition of a heat insulating member.

FIG. 8 is a diagram showing Modification Example 2 of the disposition of the heat insulating member.

FIG. 9 is a diagram showing Modification Example 3 of the disposition of the heat insulating member.

FIG. 10 is a diagram showing an aspect in which the heat insulating member is disposed between the electrical circuit component and a back surface of a middle plate and between an end portion of the middle plate and an inner surface of a side plate of an accommodation portion.

FIG. 11 is a diagram showing an aspect in which the end portion of the middle plate is attached to an inner surface of a bottom plate of the accommodation portion.

FIG. 12 is a diagram showing an aspect in which the heat insulating member is disposed between the end portion of the middle plate and the inner surface of the bottom plate of the accommodation portion.

### DETAILED DESCRIPTION

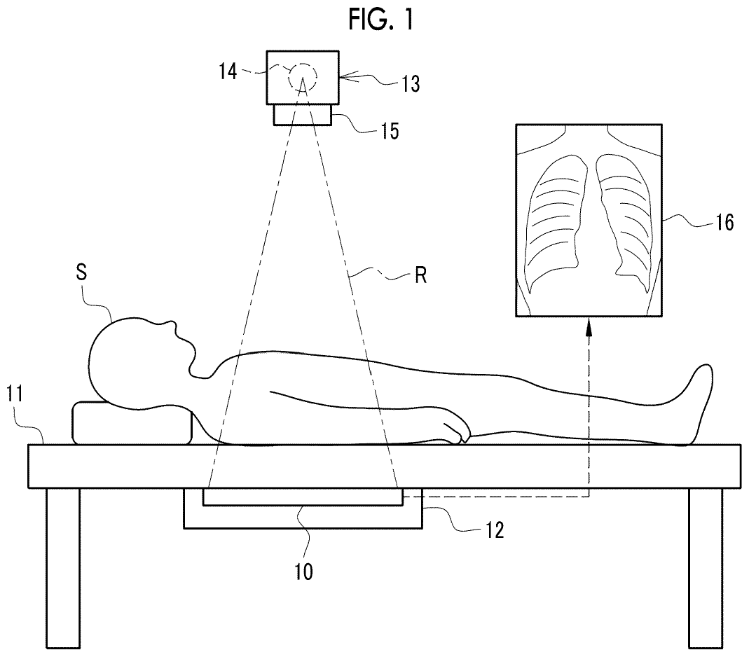

As shown in FIG. 1 as an example, a radiation image detector 10 of the present disclosure is used, for example, by being set in a holder 12 of a decubitus imaging table 11 on which a subject S lies down. A radiation source 13 is installed at a position facing the radiation image detector 10 above the decubitus imaging table 11. The radiation source 13 includes a radiation tube 14 that emits radiation R such as X-rays and γ-rays, and a collimator 15 that adjusts an irradiation field of the radiation R. The radiation image detector 10 outputs a radiation image 16 corresponding to the radiation R emitted from the radiation tube 14 and transmitted through the subject S. FIG. 1 shows a state in which a chest of the subject S is imaged by radiography. The radiation image detector 10 may be taken out from the holder 12 of the decubitus imaging table 11 and used alone. In addition, the radiation image detector 10 may be used by being set in a holder of an upright imaging table instead of or in addition to the decubitus imaging table 11.

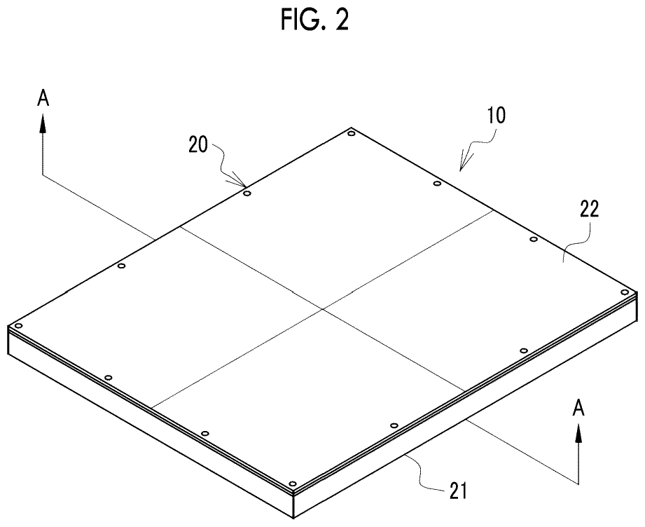

As shown in FIG. 2 as an example, the radiation image detector 10 includes a rectangular parallelepiped housing 20. The housing 20 includes an accommodation portion 21 and a radiation transmission member 22. The accommodation portion 21 is formed of, for example, a metal having a light weight, a high mechanical strength, and a high thermal conductivity, such as a magnesium alloy or an aluminum alloy. The radiation transmission member 22 is disposed on an incidence side of the radiation R. The radiation transmission member 22 is formed of a material that transmits radiation, for example, carbon fiber reinforced plastic (CFRP).

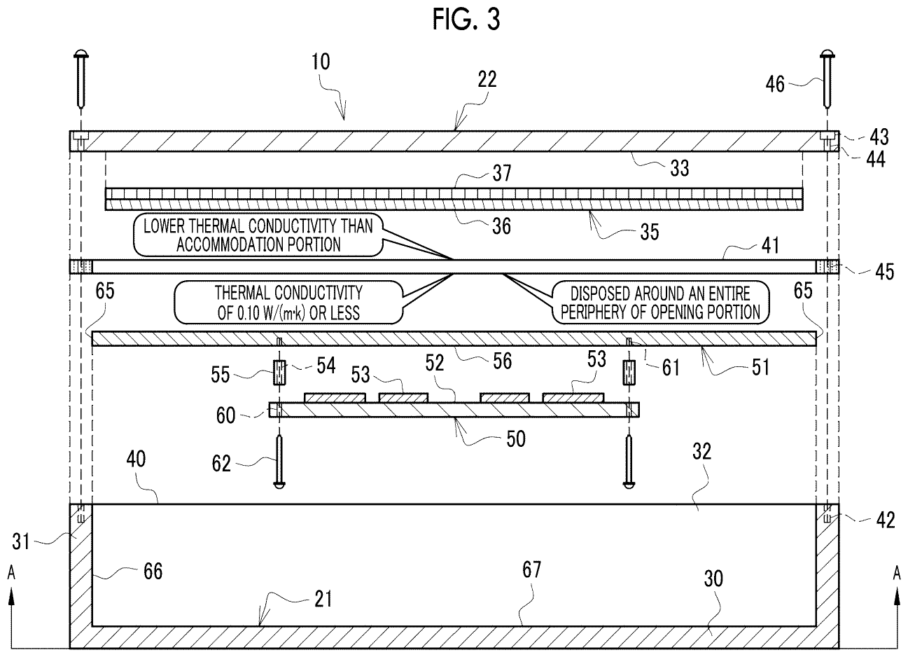

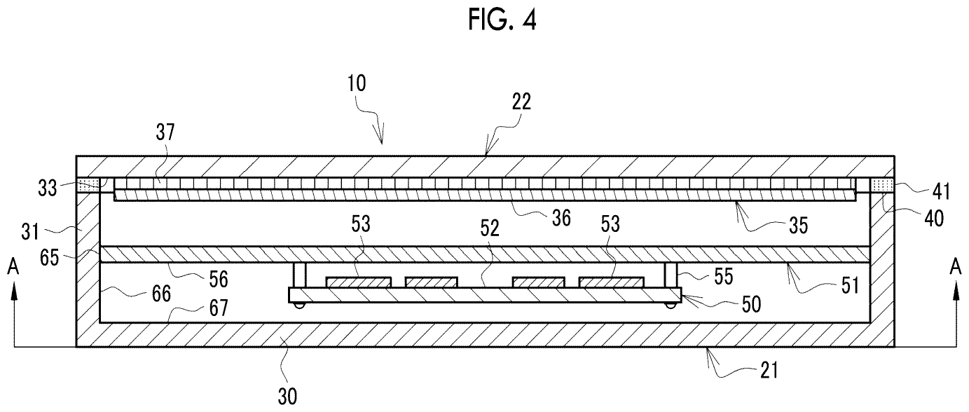

As shown in FIGS. 3 and 4, which are cross-sectional views of the radiation image detector 10 taken along line A-A of FIG. 2, the accommodation portion 21 is a U-shaped box body composed of a bottom plate 30 facing the radiation transmission member 22 and four side plates 31 bent upward by 90° from the bottom plate 30. Therefore, an opening portion 32 is formed on the incidence side of the radiation R of the accommodation portion 21. The radiation transmission member 22 closes the opening portion 32. That is, the radiation transmission member 22 constitutes a top plate (lid of the accommodation portion 21) of the housing 20.

A detection panel 35 is directly attached to a back surface 33 of the radiation transmission member 22 on a side opposite to the incidence side of the radiation R by a double-sided tape, an adhesive, or the like. The detection panel 35 is composed of a scintillator 36 and a light detection substrate 37. The light detection substrate 37 and the scintillator 36 are disposed in the order of the light detection substrate 37 and the scintillator 36 from the incidence side of the radiation R. That is, the detection panel 35 of the present example is an irradiation side sampling (ISS) method. The detection panel 35 may be a penetration side sampling (PSS) method in which the scintillator 36 and the light detection substrate 37 are disposed in this order from the incidence side of the radiation R.

The scintillator 36 has, for example, a phosphor such as thallium-activated cesium iodide (CsI:Tl) or terbium-activated gadolinium oxysulfide (GOS, Gd₂O₂S:Tb). The scintillator 36 converts the incident radiation R into visible light by the phosphor and releases the visible light. The light detection substrate 37 has a configuration in which a plurality of pixels are arranged on one thin film transistor (TFT) active matrix substrate. The light detection substrate 37 detects the visible light released from the scintillator 36 and converts the visible light into an electric signal that is the source of the radiation image 16. That is, the detection panel 35 of the present example is an indirect conversion type that detects the radiation image 16 by converting the radiation R, which is converted into visible light by the scintillator 36, into an electric signal by the light detection substrate 37. The detection panel 35 may be a direct conversion type that detects the radiation image 16 by directly converting the radiation R into an electric signal.

By directly attaching the detection panel 35 to the back surface 33 of the radiation transmission member 22, the following effects can be obtained. First, the image quality of the radiation image 16 can be improved by capturing the radiation R immediately after transmission through the radiation transmission member 22. Second, it can contribute to thinning of the housing 20. Third, a gap can be provided between the detection panel 35 and a circuit board 50 described later, and the amount of drive heat of the electrical circuit component 53 transmitted to the detection panel 35 through the air can be reduced.

A heat insulating member 41 having a rectangular frame shape following the upper end surface 40 is disposed between the upper end surface 40 of the side plate 31 of the accommodation portion 21 and the back surface 33 of the radiation transmission member 22.

In other words, the heat insulating member 41 is held in a state of being sandwiched between the upper end surface 40 of the accommodation portion 21 and the back surface 33 of the radiation transmission member 22. That is, the heat insulating member 41 is disposed around an entire periphery of the opening portion 32, and the accommodation portion 21 and the radiation transmission member 22 are completely separated from each other by the heat insulating member 41. The heat insulating member 41 has, for example, a thermal conductivity of 0.10 W/(m·K) or less at 23 °C. The heat insulating member 41 is formed of, for example, a foam material such as foam polyethylene or foam polypropylene, a resin such as polycarbonate or polyamide, or an inorganic material such as a ceramic fiber sheet. Therefore, the heat insulating member 41 has a lower thermal conductivity than the accommodation portion 21 formed of metal.

A screw hole 42 is cut at a predetermined interval at a plurality of locations on the upper end surface 40. A counterbore 43 and an insertion hole 44 are formed in a portion of the radiation transmission member 22 corresponding to the screw hole 42. In addition, an insertion hole 45 is formed in a portion of the heat insulating member 41 corresponding to the screw hole 42. A screw 46 is inserted into the counterbore 43, the insertion hole 44, and the insertion hole 45. Then, the screw 46 is screwed into the screw hole 42, so that the radiation transmission member 22 and the heat insulating member 41 are fastened and fixed to the accommodation portion 21. As a result, the detection panel 35 attached to the back surface 33 of the radiation transmission member 22 is accommodated in the accommodation portion 21. The accommodation portion 21, the radiation transmission member 22, and the heat insulating member 41 may be fixed by a double-sided tape, an adhesive, or the like instead of being fastened and fixed by the screw 46.

The accommodation portion 21 further accommodates a circuit board 50 and a middle plate 51. A plurality of electrical circuit components 53 are mounted on a surface 52 on the incidence side of the radiation R on the circuit board 50. The electrical circuit component 53 is a component of a circuit that performs drive control of the radiation image detector 10, such as an integrated circuit (IC).

The circuit board 50 is attached to a back surface 56 of the middle plate 51 on a side opposite to the incidence side of the radiation R via a plurality of cylindrical spacers 55 having the insertion hole 54. Therefore, the surface 52 of the circuit board 50 on which the electrical circuit component 53 is mounted and the back surface 56 of the middle plate 51 face each other with a spacing defined by the spacers 55.

An insertion hole 60 is formed in a portion of the circuit board 50 corresponding to the spacer 55. In addition, a screw hole 61 is formed in a portion of the back surface 56 of the middle plate 51 corresponding to the spacer 55. A screw 62 is inserted into the insertion holes 54 and 60. Then, the screw 62 is screwed into the screw hole 61, so that the circuit board 50 is fastened and fixed to the middle plate 51. The circuit board 50 and the middle plate 51 may be fixed by a double-sided tape, an adhesive, or the like instead of being fastened and fixed by the screw 62.

An end portion 65 of the middle plate 51 is attached to an inner surface 66 of the side plate 31 of the accommodation portion 21 by a double-sided tape, an adhesive, or the like. A position where the end portion 65 is attached is adjusted such that a height of the circuit board 50 from an inner surface 67 of the bottom plate 30 is a predetermined height. Therefore, the circuit board 50 is accommodated in the accommodation portion 21 in a hanging state. Although not shown, an analog/digital converter or the like that converts an analog electrical signal output from the light detection substrate 37 into a digital electrical signal is mounted on the back surface 56 of the middle plate 51.

The electrical circuit component 53 generates heat by driving. In particular, in video imaging in which the radiation image 16 is continuously captured at a predetermined frame rate (for example, 30 frames per second (FPS)), the drive heat of the electrical circuit component 53 is further increased. In a case where the drive heat of the electrical circuit component 53 is transmitted to the detection panel 35, unevenness occurs in the temperature characteristics of the detection panel 35, and the unevenness appears as an artifact in the radiation image 16.

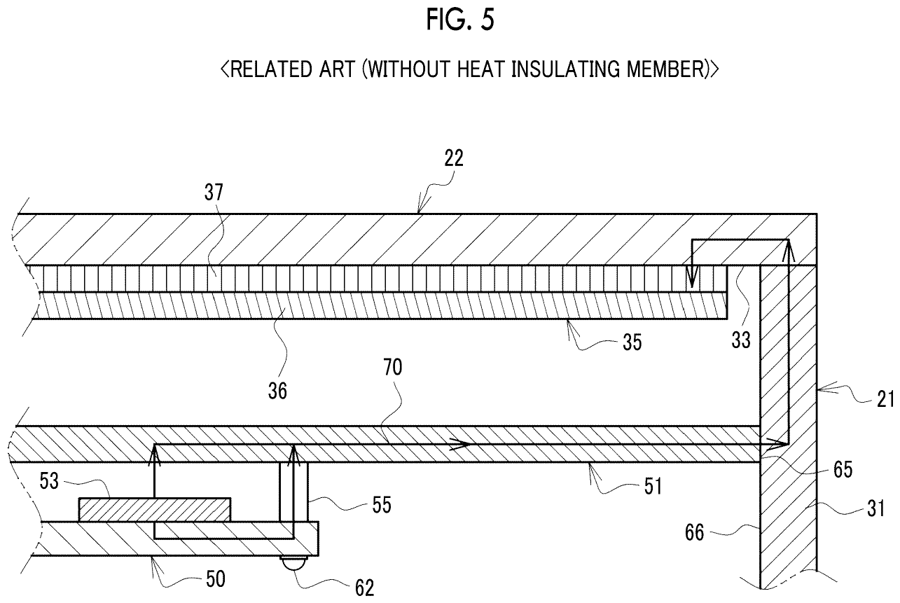

As shown in FIG. 5 as an example, in the related art in which the heat insulating member 41 is not disposed between the accommodation portion 21 and the radiation transmission member 22, the drive heat of the electrical circuit component 53 is transmitted as follows. That is, as indicated by an arrow and a reference numeral 70, the drive heat of the electrical circuit component 53 is first transmitted to the middle plate 51 through air. In addition, the drive heat of the electrical circuit component 53 is also transmitted to the middle plate 51 through the circuit board 50, the spacer 55, and the screw 62. Then, the drive heat is transmitted from the middle plate 51 to the side plate 31 of the accommodation portion 21 to which the end portion 65 is attached on the inner surface 66. Next, the drive heat is transmitted from the side plate 31 to the radiation transmission member 22, and is finally transmitted to the detection panel 35 through the back surface 33 of the radiation transmission member 22.

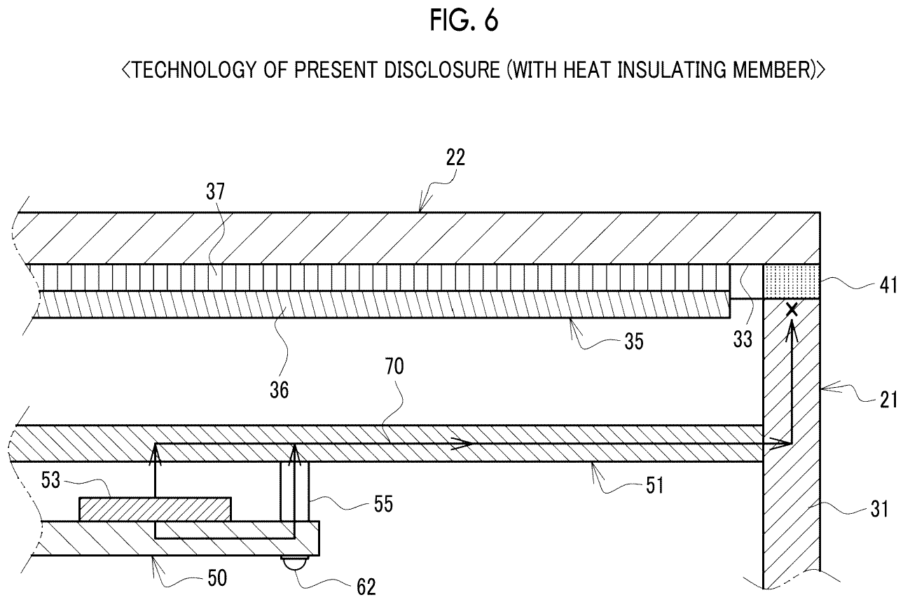

On the other hand, as shown in FIG. 6 as an example, in the technology of the present disclosure in which the heat insulating member 41 is disposed between the accommodation portion 21 and the radiation transmission member 22, the drive heat of the electrical circuit component 53 is transmitted to the side plate 31 of the accommodation portion 21, but is not transmitted to the radiation transmission member 22 from the side plate 31. That is, the drive heat of the electrical circuit component 53 transmitted to the detection panel 35 through the accommodation portion 21 and the radiation transmission member 22 is heat-insulated by the heat insulating member 41. Therefore, according to the technology of the present disclosure, it is possible to reduce the risk that the drive heat of the electrical circuit component 53 is transmitted to the detection panel 35 through the radiation transmission member 22. As a result, it is possible to suppress the deterioration in the image quality of the radiation image 16.

As can be seen by comparing FIGS. 5 and 6, the heat insulating member 41 is disposed by scraping a portion of the accommodation portion 21 that is originally the side plate 31. Therefore, the thinning of the housing 20 is not hindered by the heat insulating member 41. In addition, by disposing the heat insulating member 41 between the accommodation portion 21 and the radiation transmission member 22, it is possible to contribute to the thinning and weight reduction of the housing 20 as compared with a case in which the sheet-like heat insulating member 41 is disposed on the entire back surface 33 of the radiation transmission member 22.

The heat insulating member 41 is disposed around the entire periphery of the opening portion 32. Then, the accommodation portion 21 and the radiation transmission member 22 are completely separated from each other by the heat insulating member 41. Therefore, it is possible to further reduce the risk that the drive heat of the electrical circuit component 53 is transmitted to the detection panel 35 through the radiation transmission member 22.

The detection panel 35 is composed of a scintillator 36 that converts the radiation R into light and a light detection substrate 37 on which pixels that accumulate charges corresponding to the light are arranged. In addition, the scintillator 36 and the light detection substrate 37 are disposed in the order of the light detection substrate 37 and the scintillator 36 from the incidence side of the radiation R. The scintillator 36 is more susceptible to the influence of heat. Then, even though the heat insulating member 41 is disposed, there is a possibility that the drive heat of the electrical circuit component 53 is transmitted to the radiation transmission member 22. Therefore, in a case in which the ISS method is adopted as described above to separate the scintillator 36 from the radiation transmission member 22, it is possible to further suppress the image quality degradation of the radiation image 16.

The accommodation portion 21 further accommodates the middle plate 51. The circuit board 50 is attached to the back surface of the middle plate 51 on a side opposite to the incidence side of the radiation R. Therefore, it is possible to reduce the amount of the drive heat of the electrical circuit component 53 transmitted to the detection panel 35 through the air. That is, the middle plate 51 can function as the heat insulating member.

The end portion 65 of the middle plate 51 is attached to the inner surface 66 of the side plate 31 of the accommodation portion 21. Therefore, the middle plate 51 can be stably held in the accommodation portion 21.

The heat insulating member 41 has a lower thermal conductivity than the accommodation portion 21. Therefore, it is possible to promote heat radiation to another conduction path, such as the outside of the accommodation portion 21, while insulating the drive heat of the electrical circuit component 53 transmitted to the detection panel 35 through the accommodation portion 21 and the radiation transmission member 22 by the heat insulating member 41.

The heat insulating member 41 has a thermal conductivity of 0.10 W/(m·K) or less. Therefore, it is possible to more effectively insulate the drive heat of the electrical circuit component 53.

### Modification Example 1 of Disposition of Heat Insulating Member

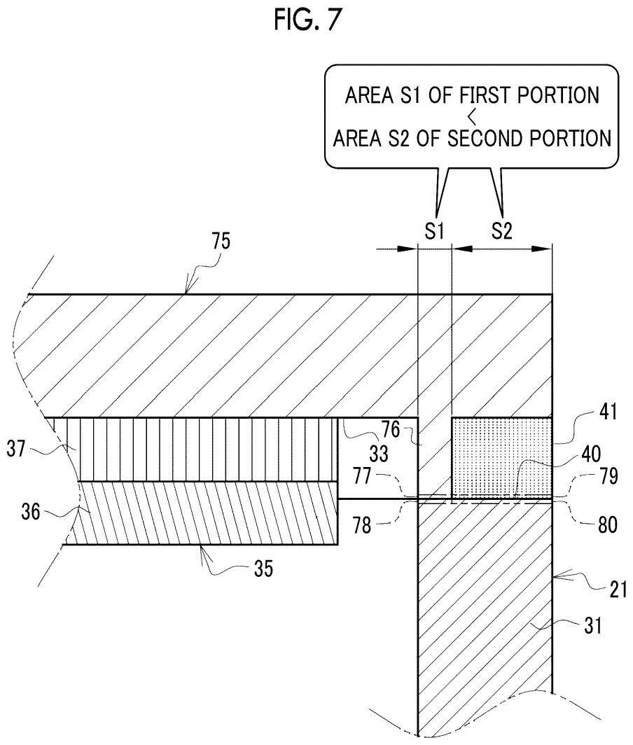

As shown in FIG. 7 as an example, in the radiation transmission member 75 of the present example, a protrusion portion 76 protruding downward is formed in a portion of the back surface 33 facing the upper end surface 40 of the side plate 31. The protrusion portion 76 is provided on the entire periphery of the radiation transmission member 75. The radiation transmission member 75 comes into contact with a first portion 78 of the accommodation portion 21 facing the radiation transmission member 75 by a first portion 77 in which the protrusion portion 76 is formed. Then, a remaining second portion 79 of the radiation transmission member 75 and a second portion 80 of the accommodation portion 21 facing the radiation transmission member 75 are separated by the heat insulating member 41. An area S1 of the first portions 77 and 78 is smaller than an area S2 of the second portions 79 and 80.

As described above, in the Modification Example 1, the first portions 77 and 78 of the accommodation portion 21 and the radiation transmission member 75 come into contact with each other, and the remaining second portions 79 and 80 are separated by the heat insulating member 41. The heat insulating member 41 is fitted into a groove-shaped portion composed of the protrusion portion 76 and the upper end surface 40. Therefore, the accommodation portion 21 and the radiation transmission member 75 can be assembled in a state in which the heat insulating member 41 is engaged with the protrusion portion 76, and the ease of manufacturing can be improved.

The heat insulating effect is reduced by the contact between the accommodation portion 21 and the radiation transmission member 75 in the first portions 77 and 78, but the area S1 of the first portions 77 and 78 is made smaller than the area S2 of the second portions 79 and 80, so that the reduction in the heat insulating effect can be minimized. It is preferable that the area S1 of the first portions 77 and 78 is smaller than ½ of the area S2 of the second portions 79 and 80 (S1<S2/2), and it is more preferable that the area S1 of the first portions 77 and 78 is smaller than ¼ of the area S2 of the second portions 79 and 80 (S1<S2/4).

### Modification Example 2 of Disposition of Heat Insulating Member

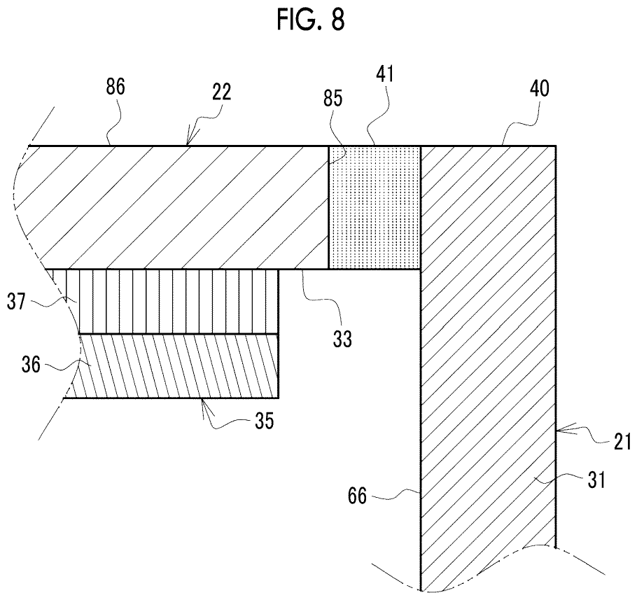

As shown in FIG. 8 as an example, in the present example, the inner surface 66 of the side plate 31 of the accommodation portion 21 and the side surface 85 of the radiation transmission member 22 are arranged to face each other such that the upper end surface 40 of the side plate 31 of the accommodation portion 21 and the surface 86 of the radiation transmission member 22 are on the same plane, instead of the upper end surface 40 of the side plate 31 of the accommodation portion 21 and the back surface 33 of the radiation transmission member 22. Then, the heat insulating member 41 is disposed between the inner surface 66 of the side plate 31 of the accommodation portion 21 and the side surface 85 of the radiation transmission member 22, instead of between the upper end surface 40 of the side plate 31 of the accommodation portion 21 and the back surface 33 of the radiation transmission member 22. Even with such a configuration, it is possible to reduce the possibility that the drive heat of the electrical circuit component 53 is transmitted to the detection panel 35 through the radiation transmission member 22.

### Modification Example 3 of Disposition of Heat Insulating Member

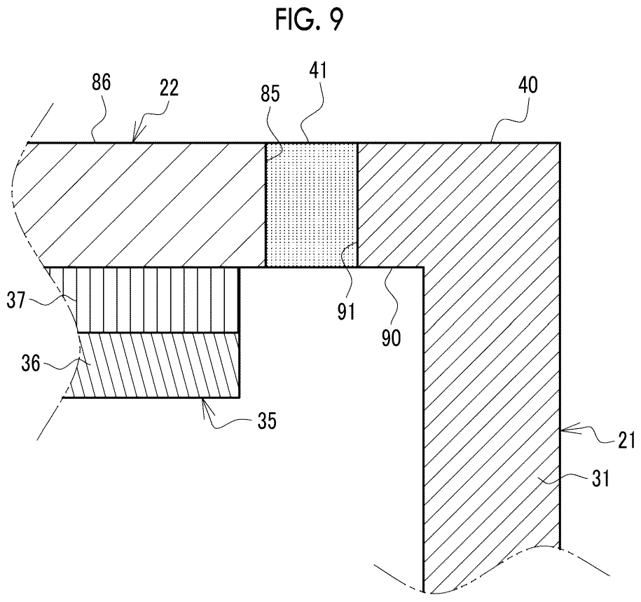

As shown in FIG. 9 as an example, in the present example, the side plate 31 of the accommodation portion 21 is bent at 90° in the horizontal direction to form a frame portion 90. Then, the end surface 91 of the frame portion 90 and the side surface 85 of the radiation transmission member 22 are arranged to face each other such that the upper end surface 40 of the side plate 31 of the accommodation portion 21 and the surface 86 of the radiation transmission member 22 are on the same plane. Further, the heat insulating member 41 is disposed between the end surface 91 of the frame portion 90 and the side surface 85 of the radiation transmission member 22. Even with such a configuration, it is possible to reduce the possibility that the drive heat of the electrical circuit component 53 is transmitted to the detection panel 35 through the radiation transmission member 22.

In Modification Example 2 shown in FIG. 8 and Modification Example 3 shown in FIG. 9, Modification Example 1 may be adopted.

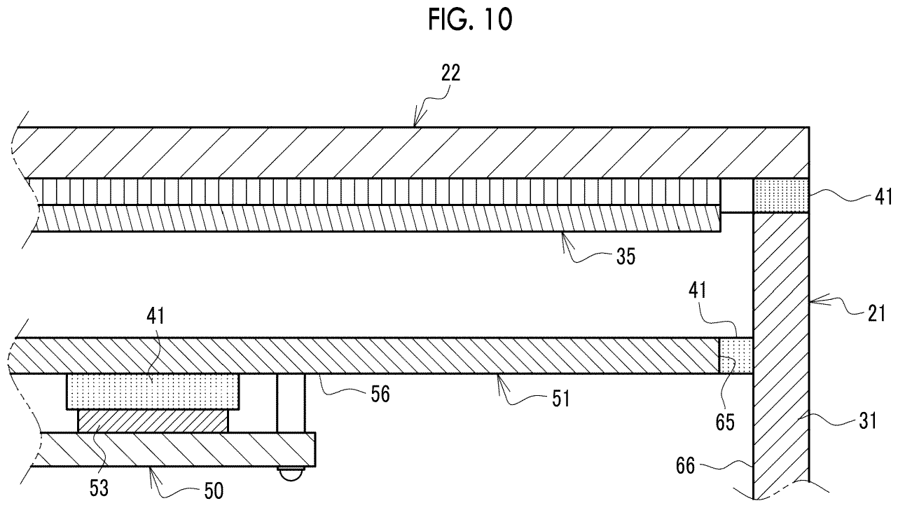

As shown in FIG. 10 as an example, the heat insulating member 41 may be disposed between the electrical circuit component 53 and the back surface 56 of the middle plate 51. In addition, the heat insulating member 41 may be disposed between the end portion 65 of the middle plate 51 and the inner surface 66 of the side plate 31 of the accommodation portion 21. The heat insulating member 41 disposed between the electrical circuit component 53 and the back surface 56 of the middle plate 51 is formed of a gel-like material such as a silicone gel or a polyurethane gel. By disposing the heat insulating member 41 between the electrical circuit component 53 and the back surface 56 of the middle plate 51, it is possible to reduce the amount of the drive heat of the electrical circuit component 53 transmitted to the middle plate 51 through the air. In addition, by disposing the heat insulating member 41 between the end portion 65 of the middle plate 51 and the inner surface 66 of the side plate 31 of the accommodation portion 21, it is possible to reduce the amount of the drive heat of the electrical circuit component 53 transmitted from the middle plate 51 to the side plate 31. As a result, it is possible to further reduce the possibility that the drive heat of the electrical circuit component 53 is transmitted to the detection panel 35 through the radiation transmission member 22.

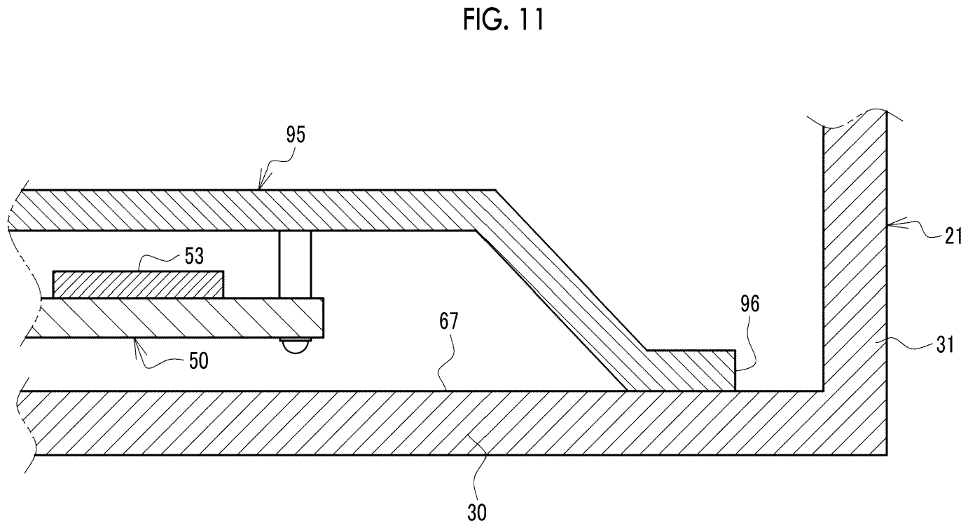

As shown in FIG. 11 as an example, a middle plate 95 may be bent toward the bottom plate 30 of the accommodation portion 21. Then, an end portion 96 of the middle plate 95 may be attached to an inner surface 67 of the bottom plate 30. The drive heat of the electrical circuit component 53 transmitted to the middle plate 95 is transmitted to the bottom plate 30 through the end portion 96. Therefore, a path through which the drive heat of the electrical circuit component 53 is dissipated can be made longer than in the above-described example in which the end portion 65 of the middle plate 51 is attached to the inner surface 66 of the side plate 31 of the accommodation portion 21. As a result, it is possible to further reduce the possibility that the drive heat of the electrical circuit component 53 is transmitted to the detection panel 35 through the radiation transmission member 22.

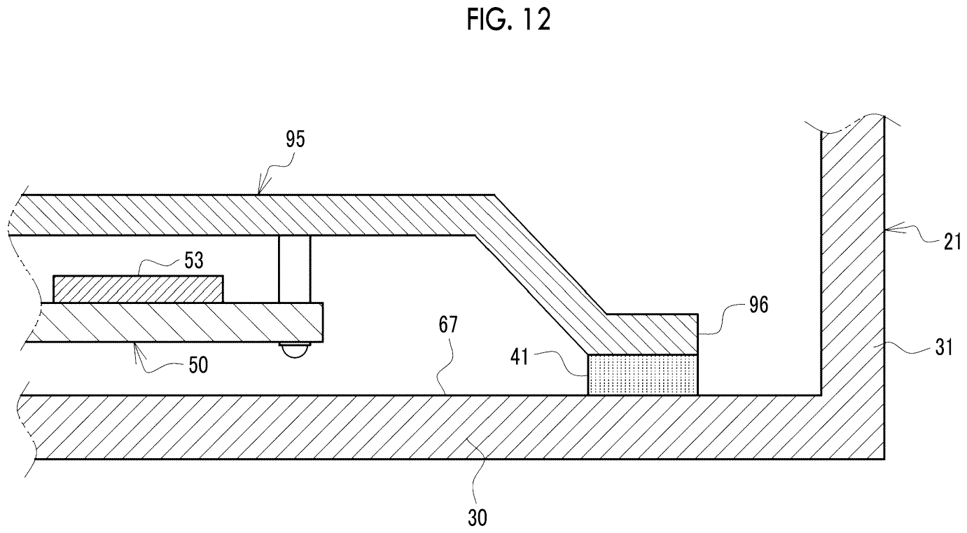

As shown in FIG. 12 as an example, the heat insulating member 41 may be disposed between the end portion 96 of the middle plate 95 and the inner surface 67 of the bottom plate 30 of the accommodation portion 21. It is possible to reduce the amount of the drive heat of the electrical circuit component 53 transmitted from the middle plate 95 to the bottom plate 30. As a result, it is possible to further reduce the possibility that the drive heat of the electrical circuit component 53 is transmitted to the detection panel 35 through the radiation transmission member 22.

The technology according to the following supplementary notes can be understood from the above description.

### Supplementary Note 1

A radiation image detector comprising:
a detection panel that detects a radiation image corresponding to radiation transmitted through a subject;
a circuit board on which an electrical circuit component is mounted;
a housing that includes an accommodation portion that accommodates the detection panel and the circuit board and in which an opening portion is formed on an incidence side of the radiation, and a radiation transmission member that closes the opening portion and transmits the radiation, the detection panel being attached to a back surface of the radiation transmission member on a side opposite to the incidence side; and
a heat insulating member that is disposed between the accommodation portion and the radiation transmission member and insulates drive heat of the electrical circuit component transmitted to the detection panel through the accommodation portion and the radiation transmission member.

### Supplementary Note 2

The radiation image detector according to Supplementary Note 1,
wherein the heat insulating member is disposed around an entire periphery of the opening portion, and
the accommodation portion and the radiation transmission member are completely separated from each other by the heat insulating member.

### Supplementary Note 3

The radiation image detector according to Supplementary Note 1,
wherein a first portion of the accommodation portion and a first portion of the radiation transmission member are in contact with each other, and a remaining second portion of the accommodation portion and a remaining second portion of the radiation transmission member are separated from each other by the heat insulating member.

### Supplementary Note 4

The radiation image detector according to Supplementary Note 3,
wherein the first portion has a smaller area than the second portion.

### Supplementary Note 5

The radiation image detector according to any one of Supplementary Notes 1 to 4,
wherein the detection panel is composed of
a scintillator that converts the radiation into light, and
a light detection substrate on which pixels that accumulate electric charges corresponding to the light are arranged, and
the light detection substrate and the scintillator are disposed in this order from the incidence side of the radiation.

### Supplementary Note 6

The radiation image detector according to any one of Supplementary Notes 1 to 5,
wherein the accommodation portion further accommodates a middle plate, and
the circuit board is attached to a back surface of the middle plate on a side opposite to the incidence side.

### Supplementary Note 7

The radiation image detector according to Supplementary Note 6,
wherein the circuit board is attached to the back surface of the middle plate via a spacer,
the electrical circuit component is mounted on a surface of the circuit board facing the back surface of the middle plate, and
the heat insulating member is disposed between the electrical circuit component and the back surface of the middle plate.

### Supplementary Note 8

The radiation image detector according to Supplementary Note 6 or 7,
wherein an end portion of the middle plate is attached to an inner surface of a side plate of the accommodation portion.

### Supplementary Note 9

The radiation image detector according to Supplementary Note 8,
wherein the heat insulating member is disposed between the end portion of the middle plate and the inner surface of the side plate.

### Supplementary Note 10

The radiation image detector according to Supplementary Note 6 or 7,
wherein an end portion of the middle plate is attached to an inner surface of a bottom plate of the accommodation portion.

### Supplementary Note 11

The radiation image detector according to Supplementary Note 10,
wherein the heat insulating member is disposed between the end portion of the middle plate and the inner surface of the bottom plate.

### Supplementary Note 12

The radiation image detector according to any one of Supplementary Notes 1 to 11,
wherein the heat insulating member has a lower thermal conductivity than the accommodation portion.

### Supplementary Note 13

The radiation image detector according to any one of Supplementary Notes 1 to 12,
wherein the heat insulating member has a thermal conductivity of 0.10 W/(m·K) or less.

The technology of the present disclosure can also appropriately combine various embodiments and/or various modification examples described above. It is needless to say that the technology of the present disclosure is not limited to each of the embodiments described above and various configurations can be employed without departing from the gist.

The description content and the illustrated content described above are detailed descriptions of portions related to the technology of the present disclosure and are merely an example of the technology of the present disclosure. For example, the above description of the configuration, the function, the operation, and the effect are the description of examples of the configuration, the function, the operation, and the effect of the parts according to the technology of the present disclosure. Accordingly, it goes without saying that unnecessary parts may be deleted, new elements may be added, or replacements may be made with respect to the above-described contents and the above-shown contents within a range that does not deviate from the gist of the technology of the present disclosure. Further, in order to avoid complication and facilitate understanding of the portion according to the technology of the present disclosure, the description related to common general knowledge not requiring special description in order to implement the technology of the present disclosure is omitted in the above description content and illustrated content.

In this specification, “A and/or B” is synonymous with “at least one of A or B”. That is, “A and/or B” means that it may be only A, only B, or a combination of A and B. In addition, in the present specification, in a case where three or more matters are expressed by being connected by “and/or”, the same concept as “A and/or B” is applied.

All documents, patent applications, and technical standards described in the present specification are incorporated in the present specification by reference to the same extent as in a case where each of the documents, the patent applications, and the technical standards are specifically and individually indicated to be incorporated by reference.

What is claimed is:

1. A radiation image detector comprising:
a detection panel that detects a radiation image corresponding to radiation transmitted through a subject;
a circuit board on which an electrical circuit component is mounted;
a housing that includes an accommodation portion that accommodates the detection panel and the circuit board and in which an opening portion is formed on an incidence side of the radiation, and a radiation transmission member that closes the opening portion and transmits the radiation, the detection panel being attached to a back surface of the radiation transmission member on a side opposite to the incidence side; and
a heat insulating member that is disposed between the accommodation portion and the radiation transmission member and insulates drive heat of the electrical circuit component transmitted to the detection panel through the accommodation portion and the radiation transmission member.

2. The radiation image detector according to claim 1,
wherein the heat insulating member is disposed around an entire periphery of the opening portion, and
the accommodation portion and the radiation transmission member are completely separated from each other by the heat insulating member.

3. The radiation image detector according to claim 1,
wherein a first portion of the accommodation portion and a first portion of the radiation transmission member are in contact with each other, and a remaining second portion of the accommodation portion and a remaining second portion of the radiation transmission member are separated from each other by the heat insulating member.

4. The radiation image detector according to claim 3,
wherein the first portion has a smaller area than the second portion.

5. The radiation image detector according to claim 1,
wherein the detection panel is composed of
a scintillator that converts the radiation into light, and
a light detection substrate on which pixels that accumulate electric charges corresponding to the light are arranged, and
the light detection substrate and the scintillator are disposed in this order from the incidence side of the radiation.

6. The radiation image detector according to claim 1,
wherein the accommodation portion further accommodates a middle plate, and
the circuit board is attached to a back surface of the middle plate on a side opposite to the incidence side.

7. The radiation image detector according to claim 6,
wherein the circuit board is attached to the back surface of the middle plate via a spacer,
the electrical circuit component is mounted on a surface of the circuit board facing the back surface of the middle plate, and
the heat insulating member is disposed between the electrical circuit component and the back surface of the middle plate.

8. The radiation image detector according to claim 6,
wherein an end portion of the middle plate is attached to an inner surface of a side plate of the accommodation portion.

9. The radiation image detector according to claim 8,
wherein the heat insulating member is disposed between the end portion of the middle plate and the inner surface of the side plate.

10. The radiation image detector according to claim 6,
wherein an end portion of the middle plate is attached to an inner surface of a bottom plate of the accommodation portion.

11. The radiation image detector according to claim 10,
wherein the heat insulating member is disposed between the end portion of the middle plate and the inner surface of the bottom plate.

12. The radiation image detector according to claim 1,
wherein the heat insulating member has a lower thermal conductivity than the accommodation portion.

13. The radiation image detector according to claim 1,
wherein the heat insulating member has a thermal conductivity of 0.10 W/(m·K) or less.
